(19) United States
(12) Patent Application Publication
(10) Pub. No.: US 2024/0199722 A1
(43) Pub. Date: Jun. 20, 2024

(54) NANOPLATFORM FOR TARGETING MACROPHAGE AND COMPOSITION FOR PREVENTION OR TREATMENT OF METASTATIC CANCER

(71) Applicant: SEOUL NATIONAL UNIVERSITY R&DB FOUNDATION, Seoul (KR)

(72) Inventors: Yun Sang LEE, Gyeonggi-do (KR); Seung Hyeok SEOK, Seoul (KR); Hongyoon CHOI, Seoul (KR); Hyung-jun IM, Seoul (KR); Ji Yong PARK, Incheon (KR); Minseok SUH, Seoul (KR); Hyewon CHUNG, Seoul (KR)

(73) Assignee: SEOUL NATIONAL UNIVERSITY R&DB FOUNDATION, Seoul (KR)

(21) Appl. No.: 18/285,877

(22) PCT Filed: Apr. 7, 2022
(86) PCT No.: PCT/KR2022/005046

§ 371 Date: Oct. 6, 2023

(30) Foreign Application Priority Data
Apr. 8, 2021 (KR) 10-1021-0046025

(51) Int. Cl. C07K 14/765; A61K 9/51; A61K 31/663; A61K 49/00; A61K 51/04; A61K 51/12; A61P 35/04

(52) U.S. Cl. CPC C07K 14/765; A61K 9/51; A61K 31/663; A61K 49/005; A61K 51/04; A61K 51/12; A61P 35/04

(57) ABSTRACT

A nanoplatform for targeting macrophages is obtained by a click chemistry reaction between albumin conjugated with an aizde (N₃) or cyclooctyne functional group and a transmitter conjugated with an azide (N₃) or cyclooctyne functional group, wherein the transmitter comprises a mannosyl group or a galactosyl group, and when the albumin is conjugated with the azide functional group, the transmitter is conjugated with the cyclooctyne functional group while, when the albumin is conjugated with the cyclooctyne functional group, the transmitter is conjugated with the azide functional group.

### [TECHNICAL FIELD]

The present disclosure relates to a nanoplatform for targeting macrophages and a composition for prevention or treatment of metastatic cancer.

### [BACKGROUND ART]

For cancer metastasis accounting for 90% of cancer-related deaths, the only way to maximize treatment efficiency in terms of cost loss and patient prognosis is to accelerate the treatment period based on early diagnosis.

Currently, main methods for diagnosing cancer metastasis may be divided into invasive and non-invasive methods, and the invasive method involves collecting samples from a specific organ through methods such as biopsy and performing histological analysis, but in secondary metastatic organs such as the lungs, rather than primary tumors or lymph nodes, there are limitations in sampling to actually determine metastasis.

A non-invasive method is to check expression of metastasis-related markers in body fluids such as blood or to continuously evaluate the presence of metastasis in the entire organ based on diagnostic images such as SPECT/CT, PET/CT or MRI, and currently, as clinical use, the most widely used imaging diagnostic methods are difficult to diagnose metastasis in the early stages, and are performed after the metastasis by the infiltration of cancer cells with at least 2 mm³ in size and approximately 900 million in number, i.e., only after metastasis has already progressed significantly, it can be determined whether it has metastasized. FIG. 1 is an MRI and FDG-PET diagnostic images taken on Day 21 after tumor transplantation, which is the early stage of metastasis in a 4T1 breast cancer tumor model and shows that it is difficult to early diagnose metastasis with the currently widely used MRI and FDG-PET diagnostic methods in clinical practice.

In addition, there have recently been attempts to diagnose metastasis at an early stage by judging biomarkers discovered based on various studies, including microRNA, through blood tests, but it is only meant to be one of indications for a degree of general metastasis progression or a prognosis and there are limitations in providing specific information such as whether a metastasis occurs, or metastatic organs, etc. An example of such a technology is disclosed in Korea Patent Application Publication No. 2019-0051364.

Meanwhile, the existence of a mechanism for promoting primary tumor growth and metastasis of macrophages, which occupy a significant portion of the tumor microenvironment, has already been suggested by numerous studies, and recently, it has been experimentally proven that these macrophages have specifically infiltrated metastatic organs in the early stage of metastasis, and then it forms a beneficial environment (metastatic niche) for cancer cells that enter later, and in particular, the possibility has been raised that metastasis can be effectively controlled through macrophage control in the early stages of metastasis.

The inventors intend to present the possibility of new diagnosis and treatment of metastatic cancer, such as macrophage targeting-based imaging diagnosis and macrophage targeting drug delivery, using a nanoplatform capable of targeting CD206, a representative marker of tumor macrophages.

### [DISCLOSURE]

### [Technical Problem]

An object of the present disclosure is to provide a nanoplatform for targeting macrophages that enables early diagnosis of metastatic cancer.

Another object of the present disclosure is to provide a composition for preventing or treating metastatic cancer that can efficiently remove macrophages and effectively inhibit tumor growth.

### [Technical Solution]

In order to achieve the above object, the present disclosure provides a nanoplatform for targeting macrophages, the nanoplatform being obtained by a click chemistry reaction between albumin conjugated with an azide (N₃) or cyclooctyne functional group and a transmitter conjugated with an azide (N₃) or cyclooctyne functional group, wherein the transmitter comprises a mannosyl group or a galactosyl group, and when the albumin is conjugated with the azide functional group, the transmitter is conjugated with the cyclooctyne functional group, and when the albumin is conjugated with the cyclooctyne functional group, the transmitter is conjugated with the azide functional group.

In the present disclosure, it is preferable that the number of the azide or cyclooctyne functional groups conjugated with the albumin is 1 to 10.

In the present disclosure, the nanoplatform may comprise 1 to 8 of mannosyl groups or galactosyl groups.

In the present disclosure, the transmitter may further comprise a chelating agent, and the chelating agent may be one or more selected from the group consisting of 1,4,7-triazacyclononane-1,4,7-triacetic acid (NOTA), 1,4, 7,10-tetraazacyclododecane-1,4,7,10-tetraacetic acid (DOTA), 3-[6,17-dihyroxy-7,10, 18,21-tetraoxo-27-[N-acetylhydroxylamino)-6, 11, 17,22-tetraazaheptaeicosane]thiourea (DFO), diethylenetriaminepentaacetic acid (DTPA), diaminedithiol (N2S2), 2-(4′-isothiocyanatobenzyl)-1,4,7-triazacyclononane-1,4,7-triacetic acid (p-SCN-Bn-NOTA), 1,4,7-triazacyclononane, 1 -glutaric acid-4,7-acetic acid (NODAGA), 2-(4′-isothiocyanatobenzyl)-1,4,7,10-tetraazacyclododecane-1,4,7,10-tetraacetic acid (p-SCN-Bn-DOTA), 1,4,8,11-tetraazacyclotetradecane-1,4,8,11-tetraacetic acid (TETA), 2-(4-isothiocyanatobenzyl)- diethylenetriaminepentaacetic acid (p-SCN-Bn-DTPA), 1-(4-isothiocyanatophenyl)-3-[6,17-dihyroxy-7,10,18,21-tetraoxo-27-[Nacetylhydroxylamino)-6,11,17,22-tetraazaheptaeicosane]thiourea (p-SCN-Bn-DFO), and hydrazinonicotinic acid (HYNIC).

In the present disclosure, the chelating agent may be labeled with a radioactive isotope and may be one or more selected from the group consisting of ³H, ¹¹C, ¹⁸F, ¹⁴Cl, ³²p, ³⁵S, ³⁶Cl, ⁴⁵Ca, ⁵¹Cr, ⁵⁷Co, ⁵⁸Co, ⁵⁹F, ⁶⁴Cu, ⁶⁷Ga, ⁶⁸Ga, ⁸⁹Zr, ⁹⁰Y, ⁹⁹Mo, ^{99m}Tc, ¹¹¹In, ¹³¹I, ¹²⁵I, ¹²⁴I, ¹²³I, ¹⁸⁶Re, ¹⁸⁸Re, ²²⁵Ac, ²¹²Pb, ^{117m}Sn, and ¹⁷⁷Lu.

In the present disclosure, the transmitter may further comprise a fluorescence material, and the fluorescence material may be one or more selected from the group consisting of Ferrodoxin NADP(+) reductase (FNR), cyanine-based fluorescent material, tetramethylrhodamine-5-maleimide (TAMRA), Flamma® fluorescent material, Evans Blue, Prussian blue, and indocyanine green (ICG).

The present disclosure also provides a composition for preventing or treating metastatic cancer, comprising the nanoplatform for targeting macrophages loaded with a bisphosphonate compound.

The present disclosure also provides a method for preventing or treating metastatic cancer, comprising administrating a composition comprising the nanoplatform for targeting macrophages loaded with a bisphosphonate compound.

The present disclosure also provides a use for the prevention or treatment of metastatic cancer of the nanoplatform for targeting macrophages loaded with a bisphosphonate compound.

In the present disclosure, the bisphosphonate compound may be selected from an alendronic acid, alendronate, cimadronate, clodronic acid, clodronate, Leo Pharmaceutical Products compound EB-1053, etidronic acid, etidronate, ibandronate, neridronate, olpadronate, pamidronate, pyridronate, risedronate, tiludronate, and zoledronate; or a pharmaceutically acceptable salt thereof; and a mixture thereof. In the present disclosure, the composition for preventing or treating metastatic

cancer, comprising a nanoplatform for targeting macrophages may comprise a bisphosphonate compound such that a molar ratio of the nanoplatform for targeting macrophages and the bisphosphonate compound is 1:1 to 1:5.

### [Advantageous Effects]

The nanoplatform for targeting macrophages according to the present disclosure

enables early diagnosis of cancer metastasis, which accounts for most of the causes of cancer-related death, thereby accelerating the timing of clinical application, and may be loaded with anticancer substances to efficiently remove macrophages and effectively suppress tumor growth.

Therefore, through the present disclosure, a targeting strategy for macrophages,

whose roles range from innate immunity to adaptive immunity, may be established not only to understand basic immune phenomena, but also to broadly apply it to alleviate and treat various intractable immune diseases, including cancer.

### [DESCRIPTION OF DRAWINGS]

FIG. 1 shows MRI and FDG-PET diagnostic images taken on Day 21 day of tumor transplantation, the early stage of metastasis, in a 4T1 breast cancer tumor model.

FIG. 2 quantifies the number of ADIBO functional groups introduced into HSA-ADIBO and the size of HSA-ADIBO.

FIG. 3 shows the results of measuring the size of a nanoplatform for targeting albumin-based macrophage by a Dynamic Light Scattering (DLS) method.

FIG. 4 shows the in-vitro verification results of am albumin-based nanoplatform for targeting macrophages.

FIG. 5 shows the results of verifying the targeting ability of the albumin-based nanoplatform for targeting macrophages, using immune shift data.

FIGS. 6 and 7 show the in vivo verification results of the albumin-based nanoplatform for targeting macrophages.

FIG. 8(a) shows the cancer metastasis diagnosis time of the albumin-based nanoplatform for targeting macrophages, compared to FDG diagnosis.

FIG. 8(b) shows a graph obtained by quantifying the results of FIG. 8(a).

FIG. 9 shows nuclear medicine imaging and quantification data for a Low meta (low metastasis) and High meta (high metastasis) models of the albumin-based nanoplatform for targeting macrophages.

FIG. 10 is a graph showing the absorbance according to a molar ratio of the albumin-based nanoplatform for targeting macrophages, and clodronates.

FIG. 11 is a table and graph showing a drug release rate over time of a clodronate-

loaded albumin-based nanoplatform for targeting macrophages.

FIG. 12 shows the results of CCK assay of a composition for preventing or treating metastatic cancer using the albumin-based nanoplatform for targeting macrophages.

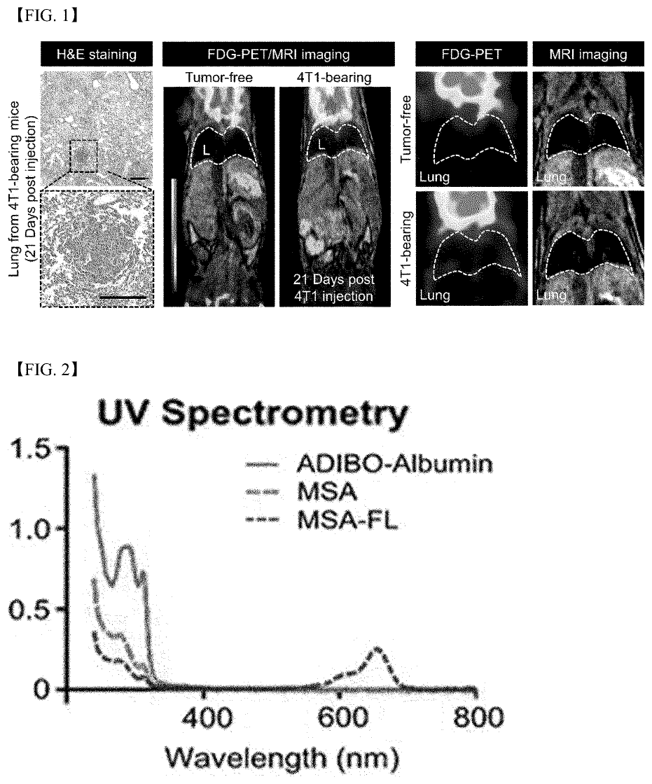

### [BEST MODE FOR INVENTION]

Unless otherwise defined, all technical and scientific terms used in the present specification have the same meaning as commonly understood by a person skilled in the art to which the present disclosure pertains. In general, the nomenclature used herein is well known and commonly used in the art.

Throughout the present specification, when a part “comprises” a certain component, it means that other components may be further included, rather than excluding other components unless specifically stated to the contrary,

One aspect of the present disclosure is an albumin-based nanoplatform for targeting macrophages, the nanoplatform being obtained by a click chemistry reaction between albumin conjugated with an azide (N₃) or cyclooctyne functional group, and a transmitter conjugated with an azide (N₃) or cyclooctyne functional group.

Albumin is one of the proteins that constitute the basic material of cells, exists in large quantities in the blood, and refers to a protein produced in the liver. The albumin has the lowest molecular weight among simple proteins that exist in nature. Serum albumin in the blood has the function of maintaining and recovering plasma volume, which prevents shock due to excessive bleeding and is used for surgery and burn treatment. It is also known to have an oxygen transport ability similar to that of hemoglobin. The albumin may comprise any albumin capable of being formulated, but may

preferably be derived from human plasma, or may be, but is not limited to, recombinant human serum albumin produced by genetic manipulation. Genetic information for albumin of the present disclosure may be obtained from known databases such as NCBI GenBank. The number of exposed amino groups (-NH2) on a surface of the albumin may

be, for example, 15 to 20.

A representative example of albumin with biocompatibility may be human serum albumin (HSA).

Azide (N₃) is a reactive group made up of three nitrogen atoms and has high reactivity, and in particular, it is known to act as an electron donor in a 1,3-dipolar cycloaddition reaction which is a type of Cu-Free click chemistry to play a role in creating a triaz-5-membered ring.

The cyclooctyne functional group is an 8-membered aliphatic ring containing a triple bond undergoing ring strain, and in particular, it is known to act as an electron acceptor in the 1,3-dipolar cycloaddition reaction, a type of Cu-Free click chemistry, to play a role in creating a triaza-5-membered ring. Due to the structural characteristics of cyclooctyne, that is, a triple bond structure undergoing ring strain, click chemistry is possible without a Cu(I) catalyst.

The cyclooctyne functional group may be, but is not necessarily limited to, one

or more selected from the group consisting of 4-cyclooctyn-1-yl

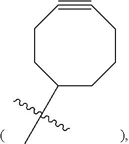 [SMILES: *C1CCC#CCCC1]

3-cyclooctyn-1-yl

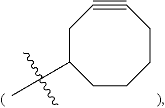 [SMILES: *C1CC#CCCCC1]

2-cyclooctyn-1-yl

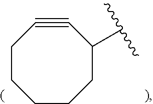 [SMILES: *C1C#CCCCCC1]

monofluorinated cyclooctyne (MOFO)

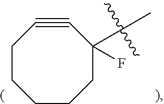 [SMILES: *C1(F)C#CCCCCC1]

difluororinated cyclooctyne (DIFO)

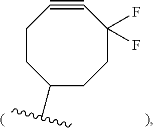 [SMILES: *C1CCC#CC(F)(F)CC1]

dimethoxyazacyclooctyne (DIMAC)

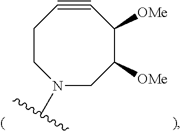 [SMILES: *N1CCC#C[C@@H](OC)[C@@H](OC)C1]

(dibenzocyclooctyne (DIBO)

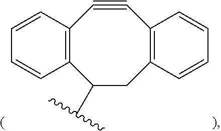 [SMILES: *C1Cc2ccccc2C#Cc2ccccc21]

azadibenzocyclooctyne (ADIBO)

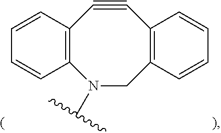 [SMILES: *N1Cc2ccccc2C#Cc2ccccc21]

and biarylazacyclooctynone (BARAC)

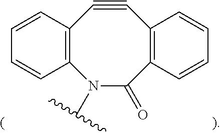 [SMILES: *N1C(=O)c2ccccc2C#Cc2ccccc21]

Albumin conjugated with the azide (N₃) or cyclooctyne functional group may be obtained by (a) dissolving albumin in phosphate-buffered saline (PBS), (b) dissolving azide-NHS or cyclooctyne-NHS in DMSO, and (c) mixing the obtained solutions and then reacting at 20 to 37° C.for 30 minutes to 1 hour.

In step (a), a phosphate buffer solution (PBS) may have a pH of 6.8 to 7.6, and preferably a pH of 7.0 to 7.4.

In step (b), the amount of DMSO used to prepare the azide-NHS solution or cyclooctyne-NHS solution may be 2% (v/v) or less of the total reaction solution.

In step (c), albumin may be mixed with azide-NHS or cyclooctyne-NHS in a molar ration of 1:1 to 1:25.

When mixing the albumin solution and the azide-NHS solution in step (c), the functional group conjugated with albumin may be the azide functional group, and when mixing the albumin solution and the cyclooctyne-NHS solution in step (c), the functional group conjugated with albumin may be the cyclooctyne functional group.

In one embodiment of the present disclosure, human serum albumin (HSA)-ADIBO was prepared by mixing the HSA solution and the ADIBO-NHS solution and reacting at 37° C.for 30 minutes.

The number of click reaction functional groups (azide or cyclooctyne functional groups) introduced to an albumin surface is preferably 10 or less, and more preferably 9 or less. In this case, when infused into the human body, the click reaction functional groups remain in the blood for a long time and may increase the possibility of ingestion into the target area. On the other hand, if the number of the click response functional groups exceeds 10, it may be ingested directly into the liver when infused into the body, thereby limiting its ingestion into other target disease areas. The number of the click reaction functional groups introduced to the albumin surface may be adjusted depending on a reaction rate between albumin and azide-NHS or cyclooctyne-NHS.

An albumin-based nanoplatform for targeting macrophages, which is an aspect of the present disclosure, is obtained by mixing a solution containing the above-described albumin conjugated with the azide (N₃) or cyclooctyne functional group, and a solution containing a transmitter conjugated with the azide (N₃) or cyclooctyne functional group and performing a click chemistry reaction, wherein the click chemistry reaction may be a copper catalyst-free (Cu-free) click chemistry reaction.

In one aspect of the present disclosure, the azide functional group used as the click chemical functional group is an electron donor, and the cyclooctyne functional group is an electron acceptor, and thus when the functional group conjugated with albumin is the azide functional group, the functional group conjugated with the transmitter is preferably the cyclooctyne functional group, and when the functional group conjugated with albumin is the cyclooctyne functional group, the functional group conjugated with the transmitter is preferably the azide functional group.

In the present disclosure, the transmitter refers to a material that is delivered into the body in combination with albumin and includes a mannosyl group or galactosyl group. The structures of the mannosyl group and galactosyl group are shown in Formulas 1 and 2 below, respectively:

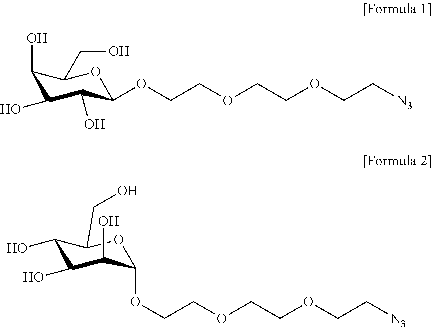

Due to this transmitter, the albumin-based nanoplatform for targeting macrophages, which is an aspect of the present disclosure, has a targeting ability for macrophages, by which drug may be delivery to macrophages, and as a result, may function as a platform for preemptive diagnosis and treatment of metastatic cancer.

Mannose receptors are known to be widely distributed in cells responsible for defense mechanisms, and the representative immune cells are Kupffer cells. When the transmitter is a mannosyl group, it acts mostly on the liver because it targets Kupffer cells, but it may also act in the blood, muscles, spleen, and lungs.

If the transmitter is a galactosyl group, it may indicate hepatobiliary excretion through the gall bladder.

The albumin-based nanoplatform for targeting macrophages may include 1 to 8 mannosyl groups or galactosyl groups, and preferably include 2 to 6 mannosyl groups or galactosyl groups.

The transmitter may further comprise one or more materials in addition to a mannosyl group or a galactosyl group, and in this case, a plurality of materials may be simultaneously subject to a click chemical reaction of albumin conjugated with the azide (N₃) or cyclooctyne functional group, and each of a plurality of materials may be sequentially subjected to the click chemical reaction.

The transmitter other than the mannosyl group or the galactosyl group may be one or more selected from the group consisting of a chelating agent and a fluorescent material, and the chelating agent may be labeled with a radioactive isotope.

A chelating agent serves to link radioactive isotopes to albumin, and for example, may be, but is not necessarily limited to, one or more selected from the group consisting of 1,4,7-triazacyclononane-1,4,7-triacetic acid (NOTA), 1,4,7,10-tetraazacyclododecane-1,4,7,10-tetraacetic acid (DOTA), 3-[6,17-dihyroxy-7,10,18,21-tetraoxo-27-[N-acetylhydroxylamino)-6,11,17,22-tetraazaheptaeicosane]thiourea (DFO), diethylenetriaminepentaacetic acid (DTPA), diaminedithiol (N2S2), 2-(4′-isothiocyanatobenzyl)-1,4,7-triazacyclononane-1,4,7-triacetic acid (p-SCN-Bn-NOTA), 1,4,7-triazacyclononane, 1 -glutaric acid-4,7-acetic acid (NODAGA), 2-(4′-isothiocyanatobenzyl)-1,4,7,10-tetraazacyclododecane-1,4,7,10-tetraacetic acid (p-SCN-Bn-DOTA), 1,4,8,11-tetraazacyclotetradecane-1,4,8,11-tetraacetic acid (TETA), 2-(4-isothiocyanatobenzyl)- diethylenetriaminepentaacetic acid (p-SCN-Bn-DTPA), 1-(4-isothiocyanatophenyl)-3-[6,17-dihyroxy-7,10,18,21-tetraoxo-27-[Nacetylhydroxylamino)-6,11,17,22-tetraazaheptaeicosane]thiourea (p-SCN-Bn-DFO), and hydrazinonicotinic acid (HYNIC).

Isotopes refers to elements that have the same atomic number but different atomic weights, and those with radioactivity are called radioactive isotopes, and is used as an important marker to generally diagnose diseases based on characteristic of radioactive attenuation by emitting gamma rays or other subatomic particles. Radioactive isotopes that may be used as markers in the present disclosure may be used without limitation as long as they are known in the art, and may be, but is not necessarily limited to, one or more selected from the group consisting of ³H, ¹¹C, ¹⁸F, ¹⁴C^{1, 32}p, ³⁵S, ³⁶C^{1, 45}Ca, ⁵¹Cr, ⁵⁷Co, ⁵⁸Co, ⁵⁹F, ⁶⁴Cu, ⁶⁷Ga, ⁶⁸Ga, ⁸⁹Zr, ⁹⁰Y, ⁹⁹Mo, ^{99m}Tc, ¹¹¹In, ¹³¹I, ¹²⁵I, ¹²⁴I, ¹²³I, ¹⁸⁶Re, ¹⁸⁸Re, ²²⁵Ac, ²¹²Pb, ^{117m}Sn, and ¹⁷⁷Lu, and preferably, the diagnostic radioisotopes such as ¹¹C, ¹⁸F, ⁶⁴Cu, ⁶⁷Ga, ⁶⁸Ga, ⁸⁹Zr, ^{99m}Tc, ¹¹¹In, and ¹²³I.

Fluorescent materials refer to a material that emits a specific wavelength of visible light for a specific wavelength, and in the present disclosure, it includes all fluorescent materials that may be used to confirm the location of the nanoplatform by binding to the albumin-based nanoplatform for targeting macrophages. Specifically, the fluorescent material that may be used as a marker in the present disclosure may be used without limitation as long as it is known in the art, and the fluorescent material such as rhodamine series including rhodamine, TAMRA, etc .; fluorescein series including fluorescein, fluorescein isothiocyanate (FITC), and fluorecein amidite (FAM) etc .; bodipy (boron-dipyrromethene) series; alexa fluor series; and cyanine series including Cy3, Cy5, Cy7, and indocyanine green, etc., may be used, but is not limited thereto.

A composition for preventing or treating metastatic cancer, which is another aspect of the present disclosure, comprises an albumin-based nanoplatform for targeting macrophages loaded with a bisphosphonate compound.

The present disclosure may also provide a method for preventing or treating metastatic cancer, comprising administrating a composition comprising a nanoplatform for targeting macrophages loaded with a bisphosphonate compound. At this time, the administration target may be a subject, specifically, a subject in need of diagnosis, prevention or treatment of metastatic cancer, and the subject may be an animal, typically a mammal.

The present disclosure may also provide a use for the prevention or treatment of metastatic cancer of the nanoplatform for targeting macrophages loaded with a bisphosphonate compound.

The bisphosphonate compound is a drug capable of selectively removing macrophages and may be selected from, but is not necessarily limited to, an alendronic acid, alendronate, cimadronate, clodronic acid, clodronate, Leo Pharmaceutical Products compound EB-1053, etidronic acid, etidronate, ibandronate, neridronate, olpadronate, pamidronate, pyridronate, risedronate, tiludronate, and zoledronate; or a pharmaceutically acceptable salt thereof; and a mixture thereof.

The albumin-based nanoplatform for targeting macrophages loaded with a bisphosphonate compound may be obtained by mixing and reacting an albumin-based nanoplatform for targeting macrophages with a bisphosphonate compound. In particular, in one embodiment of the present disclosure, it was confirmed that a bisphosphonate compound can easily bind to albumin non-specifically.

In the albumin-based nanoplatform for targeting macrophages loaded with the bisphosphonate compound according to the present disclosure, a molar ratio of the nanoplatform for targeting macrophages and the bisphosphonate compound is preferably 1:0.1 to 1:10, and more preferably 1:1 to 1:5. If the molar ratio of bisphosphonate is less than the above range, tumor suppressive activity is insufficient, and if it exceeds the above range, residual substances that no longer react with the nanoplatform are generated.

The composition for preventing or treating metastatic cancer according to the present disclosure may efficiently remove microphages even if a small amount of a toxic bisphosphonate compound is loaded, and thus has the advantage minimizing side effects due to the toxicity of the bisphosphonate compound.

Another aspect of the present disclosure, a composition for preventing or treating metastatic cancer, comprises a bisphosphonate compound loaded on an albumin-based nanoplatform to which mannosyl or galactosyl groups are introduced, and thus can efficiently remove macrophages and can effectively suppress the growth of tumor, thereby minimizing the side effects of anti-cancer therapy and maximizing its effectiveness.

In most early inflammations, M1-type macrophages (inflammatory) are more prevalent than M2-type macrophages (non-inflammatory). However, as inflammation gradually becomes chronic or cancerous, M1-type macrophages, which have the nature of attacking substances or cells that cause inflammation, are converted to M2-type macrophages or selectively attract M2-type macrophages among the macrophages flowing into the lesion, and creates an environment friendly to a cancer. The specific type of macrophage thus induced does not attack the lesion site on its own or through cytokines, allowing the chronic disease or cancer environment to evade in vivo immunity, thereby prolonging the disease. It has also been revealed that the macrophages discovered to date have not only the M1 phenotype, which simply increases immunity, but also the M2 phenotype, which suppresses the immune response and helps cancer cell growth and metastasis. Tt was confirmed that the platform of targeting macrophage according to the present disclosure may be used to predict the metastatic site in advance by targeting macrophage at the metastatic site and prevent metastasis by depleting the macrophages at that site, and in particular, in addition to accurate targets, the effect may be maximized by activating macrophages into inflammatory-type macrophages.

### EXAMPLES

Hereinafter the present disclosure will be described in more detail through examples. However, these Examples show some experimental methods and compositions to illustratively illustrate the present disclosure, and the scope of the present disclosure is not limited to these Examples.

### Preparation Example 1: Preparation of Albumin-Based Nanoplatform for Targeting Macrophage

1-1. Albumin preparation and introduction of ADIBO functional group

First, serum albumin (HSA) was used by purchasing 100 mL of human serum albumin 20% from Green Cross-Albumin Co., Ltd., a prescription drug sold in 20% liquid form at the Green Cross Medical Foundation, and dissolving it in PBS (pH 7.4) at a concentration of 5 mg/50 μl to prepare an albumin solution. In addition, ADIBO-NHS was purchased and used from FutureChem (FC-6123),

and dissolved in DMSO at a concentration of 2 mg/50 μl to prepare an ADIBO-NHS solution.

When preparing the ADIBO-NHS solution, the amount of DMSO was prepared to be not more than 2% (v/v) of the total reaction solution volume in order to ensure that the minimum amount was contained in the total reaction volume.

Next, 5 mg/50 μl of HSA solution was added to an Ependorf (EP) tube, and PBS (pH 7.4 or more) was added to make 500 μl (sample A). In addition, 2 mg/50 μl of ADIBO-NHS solution dissolved in DMSO was added to a separate EP tube according to a reaction ratio (molar ratio), and the volume was adjusted to 500 μl using PBS as above (Sample B). When preparing sample B, the solution was flowed into the EP tube along the wall of the tube and was immediately subjected to vortexing to be quickly dispersed. The samples A and B prepared above were mixed and reacted at 37° C. for 30 minutes using a tip type of thermostat to obtain an HSA-ADIBO.

The number of ADIBO functional groups introduced into HSA-ADIBO was quantified by UV-Vis spectrophotometric method, and the result was shown to be 8.4±0.32 (FIG. 2).

A binding ratio of albumin and ADIBO functional groups varies depending on the reaction ratio, and in particular, since each has a UV characteristic peak, the measured UV value may be converted to concentration according to Beer Lambert's law. UV absorbance may be expressed with the following relationship:

[Absorbance]=[Extinction coefficient]×[Concentration]×[Length]

Therefore, the concentration may be easily converted by dividing the measured absorbance by the extinction coefficient and length of the albumin or ADIBO compound. At this time, the length refers to a width of the container used when measuring

UV, and was converted to cm and substituted into the relational equation.

1-2. Binding of mannosyl group

HSA-ADIBO was dissolved in PBS, and Man-N₃(1-O-(2-(2-(2-

azidoethoxy)ethoxy)ethoxy)-alpha-D-mannopyranoside) was mixed at the molar ratio of 1:10 (HSA-ADIBO:Man-N₃), and reacted at 37° C. for 1 hour.

1-3. Binding of galactosyl group

HSA-ADIBO was dissolved in PBS, Gal-N₃(1-(2-Azidoethoxy)-beta-D-galactopyranose) was mixed at the molar ratio of 1:10 (HSA-ADIBO:Gal-N3), and reacted at 37° C. for 1 hour.

1-4. Binding of chelating agents labeled with fluorescent substances and/or radioactive isotopes

FNR648-N₃ and/or NOTA-N₃ (¹¹¹In-NOTA-N₃ or ⁶⁴Cu-NOTA-N₃) labeled with a radioactive isotope were mixed with HSA-ADIBO to which the mannosyl group was bonded, and reacted at 37° C. for 1 hour.

Next, only the albumin-based nanoplatform for targeting macrophages was separated and obtained through a PD-10 (desalting) column.

The number of mannosyl groups introduced into the albumin-based nanoplatform for targeting macrophages was found to be 4.1±0.26.

As a result of measuring the size of the albumin-based for targeting

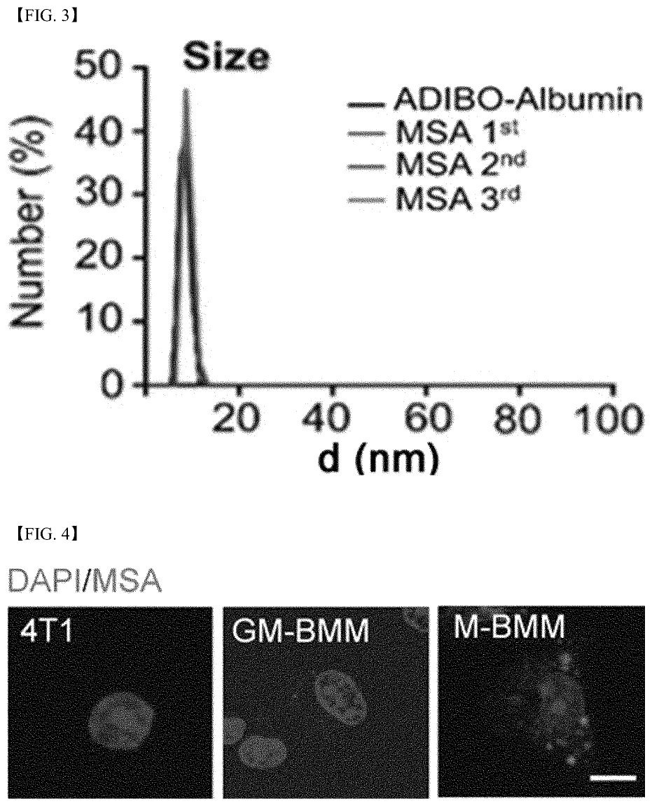

macrophages using the Dynamic Light Scattering (DLS) method, it was found to be almost the same as HSA-ADIBO before the introduction of mannosyl groups (FIG. 3).

In addition, FNR648-N₃ and/or NOTA-N₃ (¹¹¹In-NOTA-N₃ or ⁶⁴Cu-NOTA-N₃) labeled with a radioactive isotope were mixed with HSA-ADIBO to which the mannosyl group was bonded, and reacted at 37° C. for 1 hour.

Next, only the albumin-based nanoplatform for targeting macrophages was separated and obtained through a PD-10 (desalting) column.

### Experimental Example 1: In Vitro Validation of Albumin-Based Nanoplatform For Targeting Macrophage

An experiment was performed to confirm whether HSA-ADIBO conjugated with a mannosyl group, as obtained in Preparation Example 1 was targeted to tumor tissue.

The mouse breast cancer cell line 4T1, primary macrophages (GM-BMM) differentiated into GM-SCF, macrophages with relatively low CD206 expression, and primary macrophages (M-BMM) differentiated into M-SCF, cells with high CD206 expression, were prepared, respectively, and then the nanoplatform obtained in Preparation Example 1 was infused. Afterwards, the specific target image was confirmed, and the results are shown in FIG. 4.

Referring to FIG. 4, it was confirmed that the uptake of the nanoplatform was low in cells with low CD206 expression, and the uptake of the nanoplatform was high in cells with high CD206 expression, confirming that the nanoplatform according to the present disclosure is capable of targeting CD206.

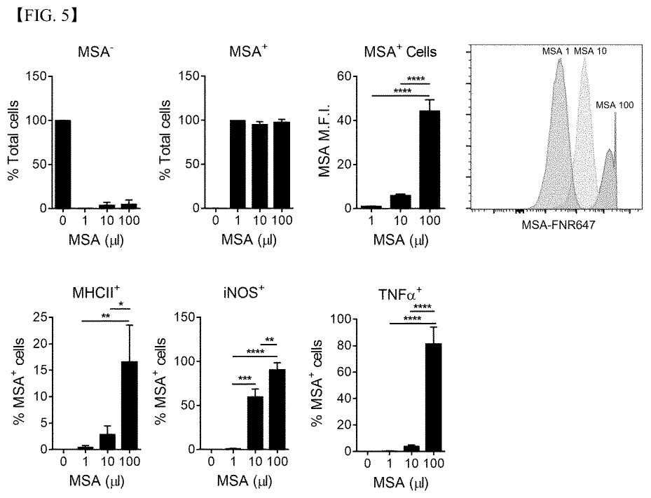

In addition, the results of an experiment to confirm the activity targeting inflammatory type of macrophage of the albumin-based nanoplatform for targeting macrophages according to the present disclosure were shown in FIG. 5.

FIG. 5 shows a graph confirming the pattern for the actual intake when the CD206 target macrophage according to the present disclosure is treated with MSA⁻ cells

(M1 polarization, macrophages with low expression of CD206) and MSA⁺ cells (M2 polarization, macrophages with high expression of CD206), respectively.

In FIG. 5, it can be seen that selective uptake was not achieved in MSA⁻ cells as a proportional relationship according to the MSA processing amount was not established, and in MSA⁺ cells, it can be seen that most cells ingested the added amount of macrophage target albumin, regardless of the processing amount. In particular, fluorescence was introduced into the albumin nanoplatform treated with MSA⁺ cells, so when the fluorescence amount according to the amount added (MSA MFI, mean fluorescence intensity) was checked, the results were proportional to the amount added and thus may be trusted to be the result of the intake pattern.

In addition, the MHCII⁺, iNOS⁺, and TNFα⁺ data in FIG. 5 are indicators of the activity of M1-type of macrophages, and when the albumin platform into which mannose was introduced to M2-type macrophages was treated at different concentrations, it was confirmed that the expression level of the indicators showing M1 activity in M2-type of macrophages increased (MSA induces macrophage phenotype shift).

### Experimental Example 2: In vivo Validation Of Albumin-Based Nanoplatform for Targeting Macrophage

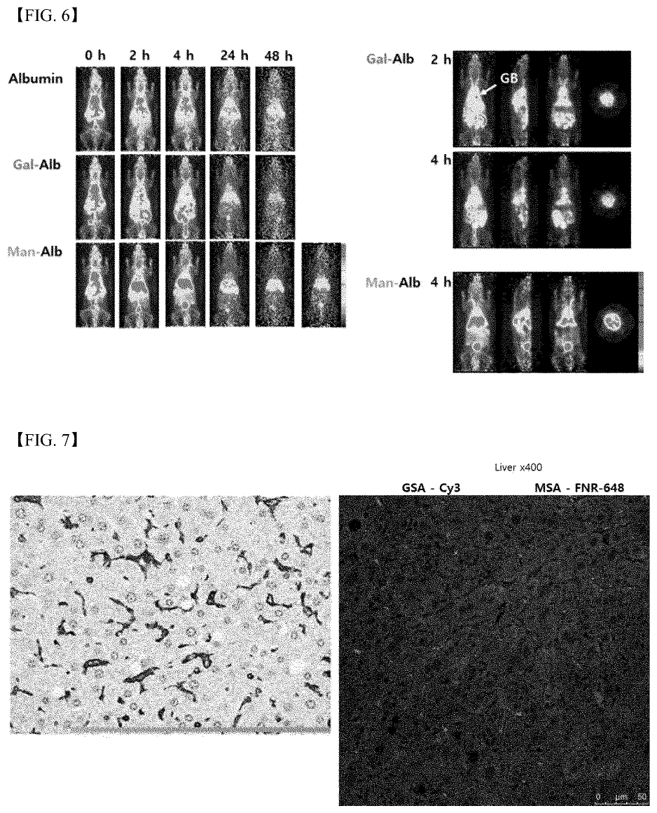

An albumin-based nanoplatform, an albumin-based nanoplatform conjugated with a mannosyl group, and an albumin-based nanoplatform conjugated with a galactosyl group were each administered into a tail vein of mice (normal group), and changes in biodistribution over each of time were observed using PET (positron emission tomography) or SPECT (single-photon emission computed tomographt), and the results are shown in FIG. 6.

As a result, when the galactosyl group is introduced, hepatobiliary excretion is shown through the gall bladder, and when the mannosyl group is introduced, strong liver intake by Kupffer cells was confirmed over a long time. Both demonstrated the versatility of albumin-based nanoplatforms.

In addition, in order to clearly confirm the morphology, the liver tissue was sectioned to obtain a fluorescence image, and the results was shown in FIG. 7. A very clear image that was identical to the actual IHC result was able to be confirmed, and this was an image obtained by removing the liver after direct injection into an animal, which was not an ex-vivo result, and it is meaningful to confirm the actual action of mannosyl and galactosyl groups in the body.

### Experimental Example 3: Verification of Possibility of Early Diagnosis of Cancer Metastasis of Albumin-Based Nanoplatform for Targeting Macrophage

In order to verify the possibility of early diagnosis of cancer metastasis of the albumin-based nanoplatform conjugated with the mannosyl group, a model that may represent cancer metastasis was reproduced and confirmed through tissue staining whether it could actually reflect metastasis.

In order to compare the albumin-based nanoplatform for targeting macrophages (MSA) obtained in Preparation Example 1 with the diagnostic method using FDG, which has been previously used in nuclear medicine imaging, 4T1-luc, wherein a fluorescent gene was introduced into the breast cancer cell line 4T1, was prepared, and cancer modeling was performed by injecting it into the femur of a mouse. This animal model is known to naturally cause metastasis to secondary organs after transplanting cancer cells into mice, and is especially known to cause metastasis to the lung area, similar to the pattern of existing breast cancer.

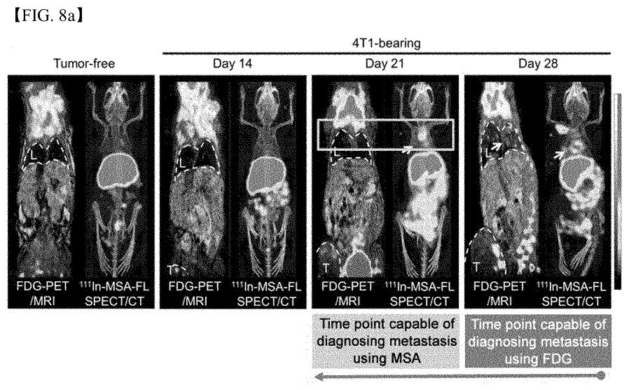

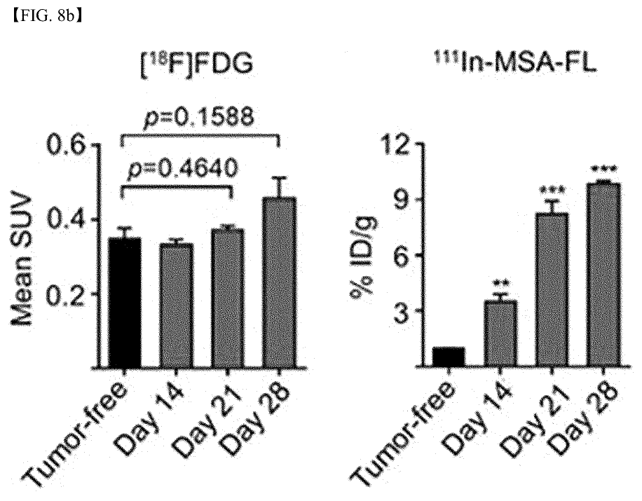

A mixture of two compounds was injected into 7, 14, 21, and 28 days-age mice after modeling, and FDG-PET images and ¹¹¹In-MSA-FL (MSA to which indium isotope and fluorescence were introduced) SPECT/CT images were obtained, and shown in FIG. 8(a).

As in the actual tissue experiment, nuclear medicine signals in the lung area could hardly be confirmed with FDG before the influx of cancer cells, that is, before metastasis occurred, but the albumin-based platform of targeting macrophage according to the present disclosure was confirmed to produce very clear images in the lung area on Day 21. Very small volume macrophage entry sites or metastasis sites that could not be identified even with the previously widely used modalities MRI and CT could be identified with very high sensitivity using nuclear medicine imaging.

These results quantified through quantification, which was an advantage of nuclear medicine imaging, were shown in FIG. 8(b). In the case of FDG, the uptake at the metastatic site showed a statistically significant difference only on Day 28, while in the case of MSA, the uptake showed a significant difference at each week, which effectively shows the influx of macrophages into the area where there is actual potential for metastasis, and this is not only a result showing that it is a nanoplatform that can be used, but it also shows the feasibility of image-based early diagnosis before metastasis of metastatic sites through quantitative data.

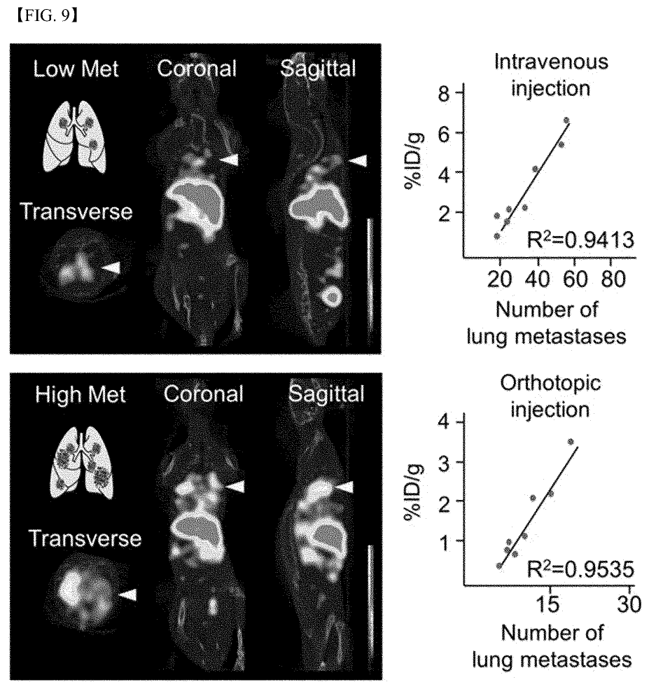

In addition, in order to directly show the correlation between the degree of uptake of the metastatic site and the degree of metastasis of the MSA thus constructed, a model wherein cancer cells were directly injected into a tail vein of mice to directly inject the cancer cells into the lungs and immediately cause metastasis, rather than using the existing model of natural metastasis from the primary cancer to the lungs, were prepared. Low meta (low metastasis) and High meta (high metastasis) models were prepared by varying the number of injected cancer cells to obtain nuclear medicine images by injecting isotopically labeled MSA, and the results of nuclear medicine quantification were shown in FIG. 9. Nuclear medicine imaging and quantification data demonstrated that there was a correlation between intake and degree of metastasis.

As a result, the diagnostic method using the albumin-based nanoplatform (MSA) for targeting macrophages was able to diagnose cancer metastasis on Day 21, but the diagnostic method using FDG was able to diagnose metastasis on Day 28, and it was confirmed that the diagnostic method using nanoplatform of the present disclosure can diagnose cancer metastasis earlier than the prior art. In particular, it was shown that the degree of metastasis can be reflected and expressed through nuclear medicine imaging and quantification.

### Preparation Example 2: Preparation of a Composition for Preventing or Treating Metastatic Cancer Using an Albumin-Based Nanoplatform for Targeting Macrophage

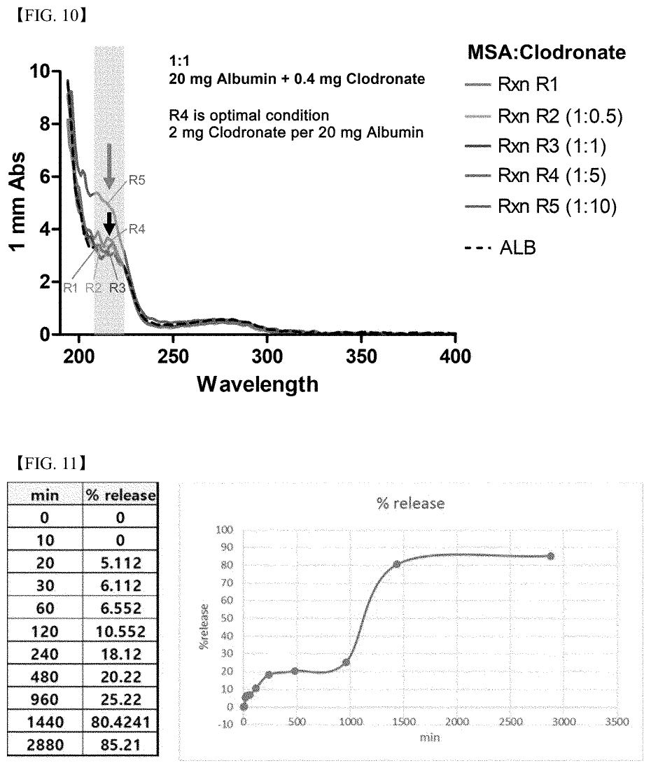

For the albumin-based nanoplatform for targeting macrophages, prepared in Preparation Example 1, clodronate, which is actually used for immune cell inhibition, was added and stirred at room temperature at the molar ratio of 1:0.25, 1:0.5, 1:1, 1:5, and 1:10, and absorbance was measured using a UV spectrometer after 30 minutes, and the results were shown in FIG. 10.

In FIG. 10, the specific peak of the bisphosphonate compound that appears at 200 to 250 nm does not appear at a molar ratio of 1:0.25 to 1:5, confirming that the entire amount is conjugated with the nanoplatform, and clodronates that do not participate in the binding at a molar ratio of 1:10 showed a UV characteristic peak.

### Experimental Example 4: Drug Release Experiment from Albumin-Based Nanoplatform Loaded with Clodronate

The 1:5 molar ratio of the clodronate-loaded albumin nanoplatform prepared in Preparation Example 2 was purified through a centrifuge, and then a sample was taken over time to calculate the amount of released clodronate.

The amount of clodronate released was confirmed by purifying the sampled clodronate/albumin complex and then checking the UV absorbance of the filtered solvent and the remaining clodronate/albumin complex to actually calculate actually released amount of clodronate, and the results were shown in FIG. 11.

In FIG. 11, the clodronate-loaded albumin nanoplatform showed about 25% release for the first 16 hours, and about 80% release after 24 hours. This result shows that the loaded bisphosphonate does not fall off immediately, but may be secreted after being delivered to the desired target like a complex, and confirmed that the nanoplatform using albumin may effectively deliver drugs to the target.

### Experimental Example 5: Drug Toxicity Experiment from Albumin-Based Nanoplatform Loaded With Clodronate

Bisphosphonate compounds, especially clodronate, have drug toxicity, so if the effective dose may be reduced, drug toxicity to other cells or organs may be reduced.

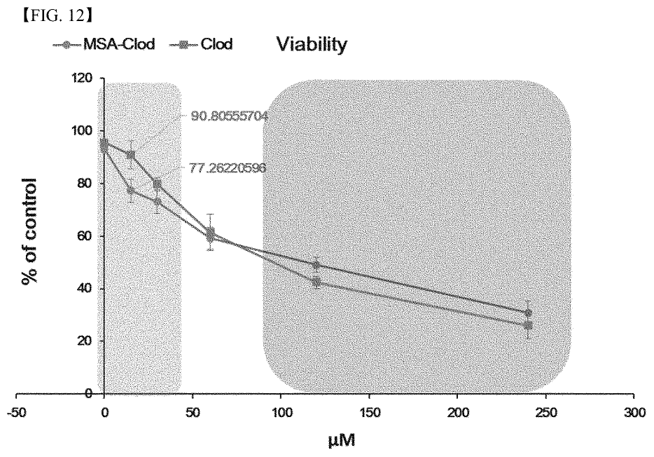

In this regard, cytotoxicity experiments of clodronate and clodronate-loaded albumin nanoplatform were performed and shown in FIG. 12.

In FIG. 12, it was confirmed that the clodronate/albumin nanoplatform showed a better drug effect at low concentrations, compared to the group treated with clodronate alone, showing that the same effect may be achieved while reducing the effective dose.

Meanwhile, at high concentrations, it was confirmed that the cell survival rate was higher than that of the group treated with clodronate alone due to the diluting effect of toxicity, generally exerted by albumin.

As the specific parts of the present disclosure have been described in detail above, it will be obvious to those skilled in the art that these specific techniques are merely preferred embodiments and do not limit the scope of the present disclosure. Therefore, it will be said that the substantial scope of the present disclosure is defined by the appended claims and their equivalents.

1. A nanoplatform for targeting macrophages, obtained by a click chemistry reaction between albumin conjugated with an azide (N₃) or cyclooctyne functional group and a transmitter conjugated with an azide (N₃) or cyclooctyne functional group,
wherein the transmitter comprises a mannosyl group or galactosyl group, and
when the albumin is conjugated with the azide functional group, the transmitter is conjugated with the cyclooctyne functional group, and when the albumin is conjugated with the cyclooctyne functional group, the transmitter is conjugated with the azide functional group.

2. The nanoplatform for targeting macrophages of claim 1, wherein the number of the azide or cyclootyne functional group conjugated with the albumin is 1 to 10.

3. The nanoplatform for targeting macrophages of claim 1, wherein the nanoplatform comprises 1 to 8 of mannosyl groups or galactosyl groups.

4. The nanoplatform for targeting macrophages of claim 1, wherein the transmitter further comprises a chelating agent, and the chelating agent is one or more selected from the group consisting of 1,4,7-triazacyclononane-1,4,7-triacetic acid (NOTA), 1,4,7,10-tetraazacyclododecane-1,4,7,10-tetraacetic acid (DOTA), 3-[6,17-dihyroxy-7,10,18,21-tetraoxo-27-[N-acetylhydroxylamino)-6,11,17,22-tetraazaheptaeicosane]thiourea (DFO), diethylenetriaminepentaacetic acid (DTPA), diaminedithiol (N2S2), 2-(4′-isothiocyanatobenzyl)-1,4,7-triazacyclononane-1,4,7-triacetic acid (p-SCN-Bn-NOTA), 1,4,7-triazacyclononane, 1-glutaric acid-4,7-acetic acid (NODAGA), 2-(4′-isothiocyanatobenzyl)-1,4,7,10-tetraazacyclododecane-1,4,7,10-tetraacetic acid (p-SCN-Bn-DOTA), 1,4,8,11-tetraazacyclotetradecane-1,4,8,11-tetraacetic acid (TETA), 2-(4-isothiocyanatobenzyl)-diethylenetriaminepentaacetic acid (p-SCN-Bn-DTPA), 1-(4-isothiocyanatophenyl)-3-[6,17-dihyroxy-7,10,18,21-tetraoxo-27-[Nacetylhydroxylamino)-6,11,17,22-tetraazaheptaeicosane]thiourea (p-SCN-Bn-DFO), and hydrazinonicotinic acid (HYNIC).

5. The nanoplatform for targeting macrophages of claim 4, wherein the chelating agent is labeled with a radioactive isotope and is one or more selected from the group consisting of ³H, ¹¹C, ¹⁸F, ¹⁴Cl, ³²P, ³⁵S, ³⁶Cl, ⁴⁵Ca, ⁵¹Cr, ⁵⁷Co, ⁵⁸Co, ⁵⁹F, ⁶⁴Cu, ⁶⁷Ga, ⁶⁸Ga, ⁸⁹Zr, ⁹⁰Y, ⁹⁹Mo, ^{99m}Tc, ¹¹¹In, ¹³¹I, ¹²⁵i, ¹²⁴I, ¹²³I, ¹⁸⁶Re, ¹⁸⁸Re, ²²⁵ Ac, ²¹²Pb, ^{117m}Sn, and ¹⁷⁷Lu.

6. The nanoplatform for targeting macrophages of claim 1, wherein the transmitter further comprises a fluorescence material, and the fluorescence material is one or more selected from the group consisting of Ferrodoxin NADP(+) reductase (FNR), cyanine-based fluorescent material, tetramethylrhodamine-5-maleimide (TAMRA), Flamma® fluorescent material, and indocyanine green (ICG).

7. A method for preventing or treating metastatic cancer, comprising administrating to a subject in need thereof a composition comprising the nanoplatform for targeting macrophages of claim 1 loaded with a bisphosphonate compound.

8. The method for preventing or treating metastatic cancer of claim 7, wherein the bisphosphonate compound is selected from an alendronic acid, alendronate, cimadronate, clodronic acid, clodronate, Leo Pharmaceutical Products compound EB-1053, etidronic acid, etidronate, ibandronate, neridronate, olpadronate, pamidronate, pyridronate, risedronate, tiludronate, and zoledronate; or a pharmaceutically acceptable salt thereof; and a mixture thereof.

9. The method for preventing or treating metastatic cancer of claim 7, wherein a molar ratio of the nanoplatform for targeting macrophages and the bisphosphonate compound is 1:1 to 1:5.
